(19) United States
(12) Plant Patent Application Publication
(10) Pub. No.: US 2017/0127589 P1
(43) Pub. Date: May 4, 2017

(54) Hop plant named 'HBC 438'

(50) ;

(71) Applicants: Eugene G. Probasco, Yakima, WA (US); Jason Perrault, Toppenish, WA (US)

(72) Inventors: Eugene G. Probasco, Yakima, WA (US); Jason Perrault, Toppenish, WA (US)

(21) Appl. No.: 15/330,748

(22) Filed: Nov. 1, 2016

Related U.S. Application Data
(60) Provisional application No. 62/285,576, filed on Nov. 2, 2015.

(51) Int. Cl. A01H 5/02

(52) U.S. Cl. PLT/236

(57) ABSTRACT

A new hop plant named ‘HBC 438’ is disclosed. ‘HBC 438’ is used for its exceptional and unique aromatic qualities.

### CROSS-REFERENCE TO RELATED APPLICATIONS

This application claims priority of co-pending provisional application Ser. No. 62/285,576 as filed Nov. 2, 2015.

### STATEMENT REGARDING FEDERALLY SPONSORED RESEARCH OR DEVELOPMENT

None

Genus and species: Humulus lupulus

Variety denomination: ‘HBC 438’

### BACKGROUND OF THE INVENTION

‘HBC 438’ is a product of a controlled breeding program carried out by the inventors in the Yakima Valley of Washington State. ‘HBC 438’ was one of several seedlings resulting from an open pollination cross made in 2004 with female parent YCR 123 (unpatented). A single plant of ‘HBC 438’ was selected in 2007, and in 2008 was expanded to 30 plants, which were planted in the area of Toppenish, Wash. The plants were observed and evaluated for several years, and in 2012 a 1 acre test plot of ‘HBC 438’ was established in the area of Toppenish, Wash. Throughout several generations of asexual propagation, ‘HBC 438’ has been observed to retain its distinctive characteristics and remain true to type.

### BRIEF DESCRIPTION OF THE PHOTOGRAPHS

FIG. 1 illustrates a mature ‘HBC 438’ hop plant grown on a trellis;

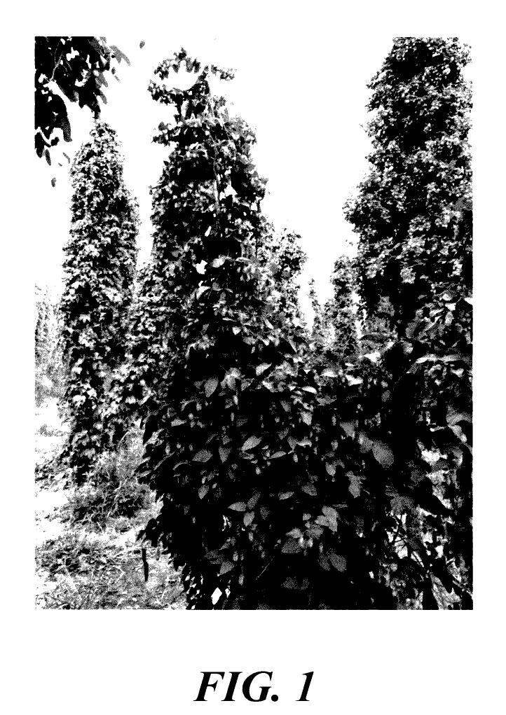

FIG. 2 illustrates whole cones and cross sections of cones of the ‘HBC 438’ hop plant;

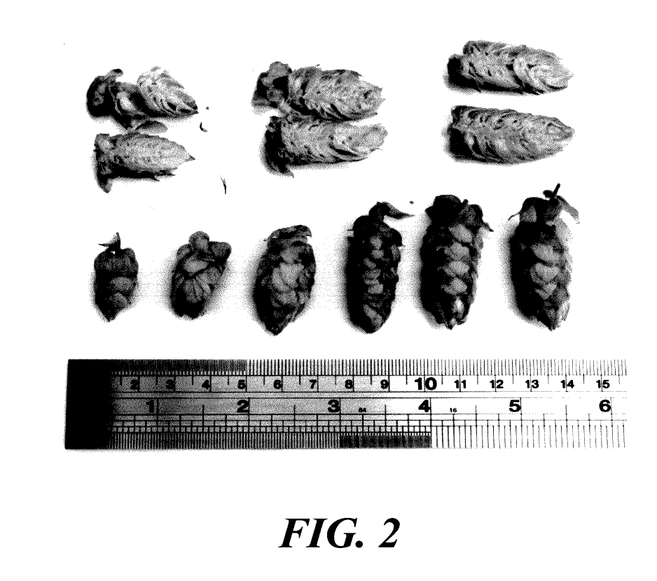

FIG. 3 illustrates the bine of a mature ‘HBC 438’ hop plant;

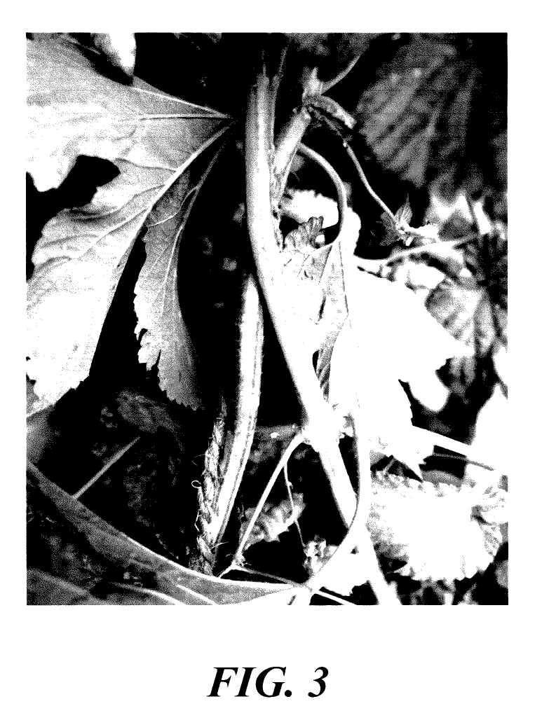

FIG. 4 illustrates cones on a mature ‘HBC 438’ hop plant;

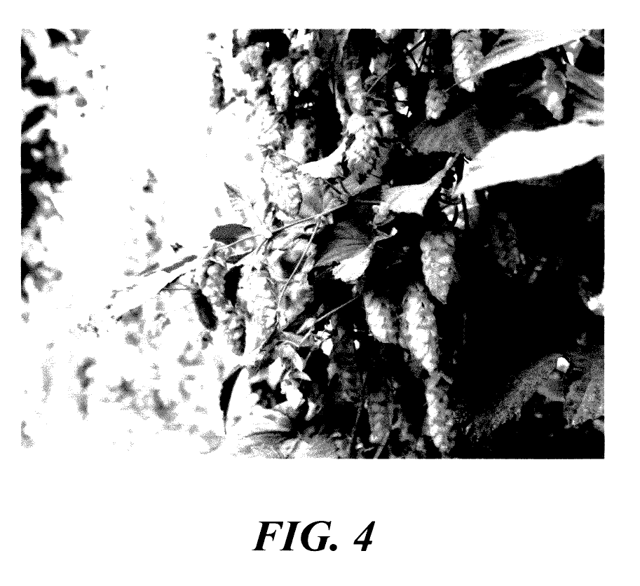

FIG. 5 illustrates leaves on a mature ‘HBC 438’ hop plant; and

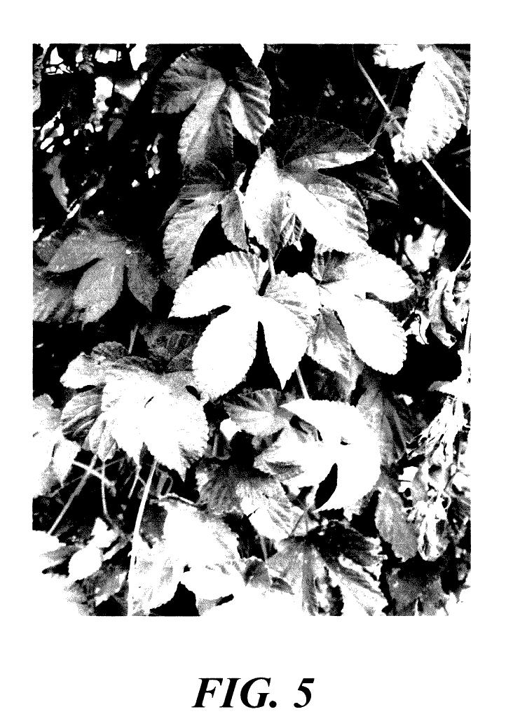

FIG. 6 illustrates leaves of the ‘HBC 438’ hop plant.

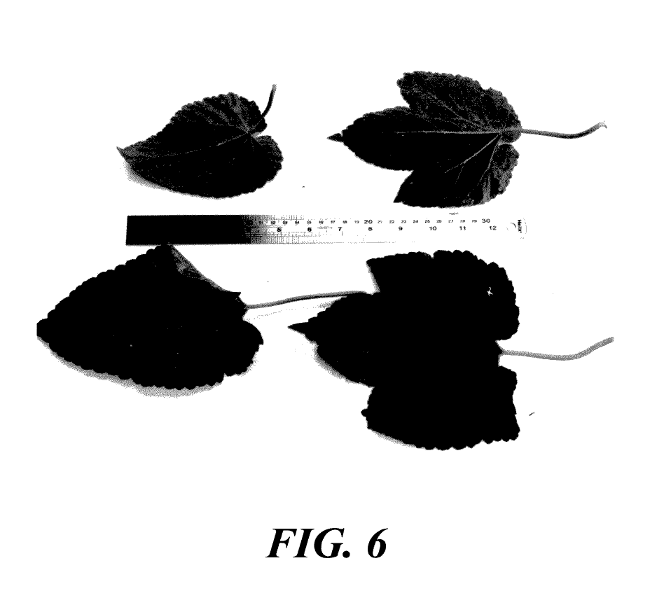

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2007-2015 growing seasons at Toppenish, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. All color references are based on The Royal Horticultural Society Color Chart.
Ploidy: Diploid (2n=2x).
Sex: Female.
Use: Brewing beer and ale.
Harvest date: September (during 2008 to 2015 growing seasons at Toppenish, Wash.).
Quality characteristics (averages):
   Alpha acid (as % of cone weight).—14.5%.
   Beta acid (as % of cone weight).—5.8%.
   Cohumulone (as % of alpha acids).—22.6%.
   Storageability (% loss).—32%.
Oil analytical characteristics (averages):
   Total oil (ml/100 g of cones).—2.95% Oil.
   A-pinene.—0.11% Total Oil.
   B-pinene.—0.90% Total Oil.
   Myrcene.—60.75% Total Oil.
   2-methyl-butyl isobutyrate.—1.92% Total Oil.
   Limonene.—0.26% Total Oil.
   Linalool.—0.41% Total Oil.
   Caryophyllene.—10.47% Total Oil.
   Farnesene.—0.61% Total Oil.
   Humulene.—10.05% Total Oil.
   Citral.—0.99% Total Oil.
   Geranyl acetate.—0.04% Total Oil.
   Citronellol.—1.19% Total Oil.
   Nerol.—0.15% Total Oil.
   Geraniol.—0.84% Total Oil.
   B-lonene.—0.01% Total Oil.
Bine:
   Color.—Yellow-Green 144A.
   Stripe present.—Yes.
   Stripe color.—Greyed-Purple 185A.
   Stipule direction.—Up.
   Bine diameter.—11 mm at base; 10 mm at nine feet; 9 mm at terminal end of eighteen feet.
Leaf:
   Arrangement.—Opposite.
   Shape.—Palmately Lobed — Cordate.
   Average length of mature leaf.—19 cm.
   Average width of mature leaf.—15 cm.
   Color of mature leaf upper surface.—Yellow-Green 147A.
   Color of mature leaf lower surface.—Yellow-Green 147B.
   Color of immature leaf upper surface.—Yellow-Green 146A.
   Color of immature leaf lower surface.—Yellow-Green 146B.
   Number of lobes.—1-3.
   Margin.—Serrate.
   Serrations per inch.—4.
   Average petiole length (mature).—9 cm.
   Petiole color at base.—Greyed-Purple 183B.
   Venation.—Palmate.
   Vein color.—Yellow-Green 146C.
Cone:
   Avg. length.—32 mm.
   Avg. diameter.—14 mm.
   Bract tip color.—Yellow-Green 144A.
   Bract base color.—Yellow-Green 145A.
   Bracteole color.—Yellow-Green 144A.
   Cone shape.—Ovate.
   Bract shape.—Ovate.
   Bract tip shape.—Acuminate-Mucronate.
   Bract tip position.—Loosely Appressed — Recurved.
   Bracteole shape.—Ovate.

1. What is claimed is a new and distinct hop plant as shown and described herein.
